(19) United States
(12) Patent Application Publication
(10) Pub. No.: US 2007/0031368 A1
(43) Pub. Date: Feb. 8, 2007

(54) Chemokine conjugates

(76) Inventors: Narendra Bam, King of Prussia, PA (US); Andrew G. King, Collegeville, PA (US); Ping-Yang Yeh, King of Prussia, PA (US); Charles Davis, Collegeville, PA (US)

Correspondence Address: GLAXOSMITHKLINE;Corporate Intellectual Property - UW2220, P.O. Box 1539, King of Prussia, PA, 19406-0939, US

(21) Appl. No.: 11/580,336

(22) Filed: Oct. 12, 2006

Related U.S. Application Data
(63) Continuation of application No. 10/312,095, filed on Dec. 20, 2002.
(60) Provisional application No. 60/215,592, filed on Jun. 30, 2000.
(60) Provisional application No. 60/252,058, filed on Nov. 20, 2000.

(51) Int. Cl. A61K 38/19

(52) U.S. Cl. 424/78.27; 424/85.1

(57) ABSTRACT

Low molecular weight polypeptides with chemokine activity were conjugated to water-soluble polymers and retain biological activity. These conjugated chemokines demonstrate enhanced and unexpected biological properties when compared to unconjugated chemokines.

### CROSS-REFERENCE TO RELATED APPLICATIONS

This is a continuation of 10/312,095 filed Dec. 20, 2002 (now pending) which is a 371 application of PCT/US01/21356, filed Jun. 29, 2001, which claims benefit of United States Provisional Applications Nos. 60/215,592, filed Jun. 30, 2000 and 60/252,058, filed Nov. 20, 2000.

### FIELD OF THE INVENTION

The instant invention relates to the field of protein conjugation. More specifically, the instant invention pertains to conjugation of water-soluble polymers to polypeptides with chemokine activity.

### BACKGROUND OF THE INVENTION

Covalent attachment of biologically active compounds to water-soluble polymers is one method for alteration and control of biodistribution, pharmacokinetics and often toxicity for these compounds (Duncan, R. and Kopecek, J. (1 984) Adv. Polym. Sci. 57:53-101). Many water-soluble polymers have been used to achieve these effects, such as poly(sialic acid), dextran, poly(N-(2-hydroxypropyl)methacrylamide) (PHPMA), poly(N-vinylpyrrolidone) (PVP), poly(vinyl alcohol) (PVA), poly(ethylene glycol-co-propylene glycol), poly(N-acryloyl morpholine (PAcM), and poly(ethylene glycol) (PEG) (Powell, G. M. (1980) Polyethylene glycol. In R.L. Davidson (Ed.) Handbook of water Soluble Gums and resins. McGraw-Hill, New York, chapter 18). PEG possess an idea set of properties: very low toxicity (Pang, S. N. J. (1993) J Am. Coll. Toxicol. 12: 429-456) excellent solubility in aqueous solution (Powell, supra), low immunogenicity and antigenicity (Dreborg, S. and Akerblom, E. B. (1990) Crit. Rev. Ther. Drug Carrier Syst. 6: 315-365). PEG-conjugated or “PEGylated” protein therapeutics, containing single or multiple chains of polyethylene glycol on the protein, have been described in the scientific literature (Clark, R. et al. (1996) J Biol. Chem. 271: 21969-21977; Hershfield, M. S. (1997) Biochemistry and immunology of poly(ethylene glycol)-modified adenosine deaminase (PEG-ADA). In J. M. Harris and S. Zalipsky (Eds) Poly(ethylene glycol): Chemistry and Biological Applications. American Chemical Society, Washington, D.C., p145-154; Olson, K. et al. (1997) Preparation and characterization of poly(ethylene glycol)ylated human growth hormone antagonist. In J. M. Harris and S. Zalipsky (Eds) Poly(ethylene glycol): Chemistry and Biological Applications. American Chemical Society, Washington, D.C., p170-181).

Conjugated proteins have numerous advantages over their unmodified counterparts. For example, PEG-modification has extended the plasma half-life of many proteins (Francis, G. E. et al. (1992) PEG-modified proteins. In: Stability of Protein Pharmaceuticals: in vivo Pathways of Degradation and Strategies for Protein Stabilization (ed by T. J. Ahern and M. manning). Plenum Press, New York). The basis for this increase involves several factors. The increased size of the PEG-modified conjugate reduces the glomerular filtration when the 70 kD threshold is exceeded (Futertges, F. and Abuchowski, A. (1990) J Controlled Release 11: 139-148). There is also reduced clearance by the reticuloendothelial system via both carbohydrate receptors and protein-receptor interactions (Beauchamp, C. O. et al. (1983) Anal. Biochem. 131: 25-33). Reduced proteolysis (Chiu, H. C. et al. (1994) J. Bioact. Comp. Polym. 9: 388-410) may also contribute to an enhanced half-life. Antigenicity and immunogenicity are also reduced (Nucci, M. L. et al. (1991) Adv. Drug DeL Rev. 6: 133-151) and this accounts for reduction in life-threatening reactions after repeated dosing. The combination of all these factors leads to increased bioavailability in vivo (Katre, N. V. et al. (1987) PNAS USA 84:1487-1491; Hershfield, M. S. et al. (1987) New England Journal ofMedicine 316: 589-596) and this is potentially very important in the use of PEG-chemokine adducts as pharmacological agents. Dose can be reduced (to alleviate toxicity) and more convenient schedule of dosing can be developed.

Members of the intercrine or chemokine family are basic heparin-binding polypeptides which have four cysteine residues which form two disulfide bridges. All these proteins which have been functionally characterized appear to be involved in proinflammatory and/or restorative functions. As such, these molecules are anticipated to have therapeutic potential in bone marrow transplantation and the treatment of infections, cancer, myelopoietic dysfunction, graft versus host disease, and autoimmune diseases (for a recent review, see Rollins, B. J. (1997) Blood 90(3):909-928).

The chemokine family can be divided into two subfamilies, the CXC and CC chemokines, based on whether the first two cysteine residues in a conserved motif are adjacent to each other or are separated by an intervening residue, respectively, and based on their chromosomal location. The CXC subfamily members are potent chemoattractants and activators of neutrophils, but not monocytes. In contrast, members of the chemokine CC subfamily are chemoattractants for monocytes, but not neutrophils.

Recently, it has been found that a number of the biomolecules identified above, as well as additional agents, can induce the mobilization of hematopoietic stem cells. The availability of recombinant cytokines and other regulatory biomolecules coupled with the use of hematopoietic stem cell support have resulted in the widespread application of high-dose chemotherapy regimens designed to improve the success of cancer therapy. While the use of these hematopoietic stem cell transplantation techniques looks promising, multiple apheresis procedures are required to harvest sufficient stem cells for successful engraftment to treat severe myelosuppression (see, e.g., Bensinger et al. (1993) Blood 81:3158 and Haas et al. (1994) Sem. in Oncology 21:19).

In cancer patients, neutropenia (less than 0.5×10⁹ neutrophils/L) is the most significant risk factor for infection following chemotherapy, and infection remains a major cause of morbidity and mortality. Febrile neutropenia is generally defined as a temperature of greater than 38.1° C. of unknown origin without clinically or microbiologically documented infection, and which lasts for four hours as determined by two readings, and an absolute neutrophil count less than 0.5×10⁹/L, which lasts for twenty-four hours, as determined by at least two readings. Likewise, chemotherapy-induced severe thrombocytopenia (less than 10×10⁹ platelets/L) is a significant side effect associated with some chemotherapeutic regimens. Besides the use of empiric broad-spectrum antibiotics, no treatment has been shown to significantly affect the outcome of chemotherapy-induced febrile neutropenia. Many chemotherapy regimens are associated with variable periods of myelosuppression. Until recently, there was no way of overcoming the problems caused by chemotherapy-induced myelosuppression other than dose reduction.

The addition of colony stimulating factors (CSFs) to myleosuppressive chemotherapy regimens can result in the reduction of the incidence of infection, hospitalization, and antibiotic therapy. The reduction in toxicity allows for maintenance of dose-density or dose-intensification of chemotherapy which will ideally result in improved response rates to chemotherapy. An agent which is more effective than the CSFs in preventing and/or reducing the severity and duration of chemotherapy-induced neutropenia and also prevented or reduced the severity and duration of thrombocytopenia could offer significant benefits by reducing the incidence and severity of infections/bleeding episodes and by allowing optimum delivery of chemotherapy (with the potential for improved response to cancer therapy).

Thus, despite these significant advances and the availability of certain regulatory biomolecules, delayed recovery of hematopoiesis remains an important source of morbidity and mortality for myelosuppressed patients. There remains a need in the art for methods of enhancing the bioactivity of chemokines to enable their efficient use as therapeutic or pharmaceutical products. There also exists a continuing need in the art for additional compositions and methods to enhance hematopoietic protection and recovery, particularly in cases of chemotherapy associated myelosuppression.

### SUMMARY OF THE INVENTION

The instant invention pertains to a biologically active composition comprising a polypeptide covalently conjugated to a water-soluble polymer wherein the polypeptide is a chemokine or a biologically active variant or derivative thereof. Preferred is a CXC chemokine, particularly the chemokine referred to herein as GroB. Most preferred is a truncated form of GroB referred to herein as GroB-t. The amino acid sequence of GroB-t is set forth in SEQ ID NO:2.

Also preferred are compositions wherein the water-soluble polymer is a member selected from the group consisting of polyethylene glycol homopolymers, polypropylene glycol homopolymers, poly(N-vinylpyrrolidone), poly(vinyl alcohol), poly(ethylene glycol-co-propylene glycol), poly(N-2-(hydroxypropyl)methacrylamide), and poly(sialic acid). These polymers may be unsubstituted or substituted at one end with an alkyl group. Particularly preferred compositions are those wherein the water-soluble polymer is a polyethylene glycol homopolymer. Most preferred are compositions wherein the polyethylene glycol homopolymer is linear.

Also preferred are compositions comprising a chemokine covalently conjugated to a water-soluble polymer and a second biologically active molecule comprising a hematopoetic growth factor. Examples of second biologically active molecules include G-CSF, GM-CSF, M-CSF, IL-3, TPO and FLT-3, as well as derivatives of these molecules, including muteins and conjugates thereof.

A further aspect of the instant invention is a method of treating myelosuppression in a patient by administering an effective dose of a biologically active composition comprising a polypeptide covalently conjugated to a water-soluble polymer wherein the polypeptide is a chemokine or a biologically active derivative thereof.

Yet a further embodiment of the instant invention is a method of enhancing the microbicidal activity of phagocytic cells in a subject by administering an effective dose of a biologically active composition comprising a polypeptide covalently conjugated to a water-soluble polymer wherein the polypeptide is a chemokine or a biologically active variant or derivative thereof.

Still a further embodiment of the instant invention is a method of mobilizing hematopoietic stem cells of a subject by administering an effective dose of a biologically active composition comprising a polypeptide covalently conjugated to a water-soluble polymer wherein the polypeptide is a chemokine or a biologically active derivative thereof.

A further aspect of the instant invention is a method of treating chemotherapy—or radiation-induced cytopenia in a patient by administering an effective dose of a biologically active composition comprising a polypeptide covalently conjugated to a water-soluble polymer wherein the polypeptide is a chemokine or a biologically active derivative thereof.

### BRIEF DESCRIPTION OF THE FIGURES

FIG. 1 is an SDS-PAGE gel scan of a series of samples from a PEGylation experiment with GroB-t.

FIG. 2 is an RP-HPLC profile of mixture of un-modified GroB-t, mono-PEGylated GroB-t, and di-PEGylated GroB-t.

FIG. 3 shows MALDI-TOF mass spectrometry results of purified mono-PEGylated GroB-t with non-modified GroB-t as the reference standard.

FIG. 4 shows peptide mapping results of non-PEGylated and mono-PEGylated GroB-t with 5K PEG following Glu-C digestion.

FIG. 5 shows the four predicted peptide fragments that are generated as a result of Glu-C digestion of GroB-t. Triangles indicate Glu-C digestion sites. Cysteine residues are underlined.

FIG. 6 shows the eleven predicted peptide fragments that are generated as a result of trypsin digestion of GroB-t. Triangles indicate trypsin digestion sites. Cysteine residues are underlined.

FIG. 7 presents data demonstrating a persistent increase in neutrophil counts in blood obtained from mice treated with PEGylated GroB-t.

FIG. 8 presents data demonstrating the increased and persistent bactercidal activity of neutrophils obtained from PEGylated GroB-t-treated animals versus neutrophils obtained from animals treated with non-PEGylated GroB-t.

FIG. 9 presents data on neutrophil counts from PEGylated GroB-t-treated animals versus neutrophil counts obtained from animals treated with non-PEGylated GroB-t.

FIG. 10 presents data comparing the intravenous pharmacokinetics of PEGylated GroB-t and unmodified GroB-t in male Sprague-Dawley rats.

FIG. 11 presents data comparing the intravenous and subcutaneous pharmacokinetics of PEGylated GroB-t in male Sprague-Dawley rats.

FIG. 12 presents data comparing the subcutaneous pharmacokinetics of PEGylated GroB-t and unmodified GroB-t in male Sprague-Dawley rats.

### DETAILED DESCRIPTION OF THE INVENTION

The present invention provides a composition comprising a polypeptide, specifically a chemokine, wherein the polypeptide is conjugated to a water-soluble polymer. The instant conjugated polypeptide demonstrates unexpected biological properties as compared to the corresponding unconjugated polypeptide. The present invention also provides methods for the treatment of hematopoiesis or lymphatic disorders, inflammation, and cancer, and, preferably, congenital cytopenias, radiation-induced cytopenia, chemotherapy-induced cytopenia (e.g. neutropenia, thrombocytopenia, anemia), hereinafter referred to as “the Diseases”, amongst others. In a further aspect, the invention relates to mobilization of hematopoietic precursor cells into the peripheral blood, their harvest, and utilization in patients requiring stem cell transplantation. The instant composition is especially useful for the treatment of myelosuppression or symptoms thereof, including chemotherapy-induced neutropenia, by mobilizing hematopoietic stem cells from the bone marrow into the peripheral blood using the composition described herein, or alternatively, by enhancing the microbicidal activity of phagocytic cells in a treated subject.

As used herein, the term “chemokine” refers to a member of a group of art-recognized proteins that act as chemoattractants for host defense effector cells such as neutrophils, monocytes and lymphocytes (see, for example, Rollins, B. J. (1997) Blood 90(3):909-928, and Baggiolini, M. (1998) Nature 392:565-568). Preferred are the “CXC” class of chemokines which includes IL-8, KC, GroA, GroB, GroG, ENA-78, GCP-2, CTAP-III, B-Thromboglobulin, NAP-2, Platlet factor 4, IP-10, MIG, SDF-1alpha and SDF-1beta. More preferred are GroA, GroB and its murine homolog, KC, and GroG. Most preferred is GroB, also known as MIP-2B. “Chemokine”, as used herein, also includes modified chemokines, including desamino proteins characterized by the elimination of between about two to about eight amino acids at the amino terminus of the mature protein. Most preferably, the modified chemokines are characterized by removal of the first four amino acids at the amino terminus. Optionally, particularly when expressed recombinantly, the desamino chemokines useful in the instant invention may contain an inserted amino terminal (N-terminal) methionine residue. The N-terminal methionine which is inserted into the protein for expression purposes, may be cleaved, either during the processing of the protein by a host cell or synthetically, using known techniques. Alternatively, if so desired, this amino acid may be cleaved through enzyme digestion or other known means.

The term “hematopoietic” cells herein refers to fully differentiated cells such as erythrocytes, granulocytes, monocytes, megakaryocytes and lymphoid cells such as T-cells and B-cells. It also encompasses the hematopoietic progenitors/stem cells from which these cells develop, such as CFU-GEMM (colony forming unit-granulocyte-erythrocyte-megakaryocyte-monocyte), CFU-GM (colony forming unit-granulocyte-monocyte), CFU-E (colony forming unit-erythrocyte), BFU-E (burst forming unit-erythrocyte), CFU-G (colony forming unit-granulocyte), CFU-eo (colony forming unit-eosinophil), and CFU-Meg (colony forming unit-megakaryocyte). The term hematopoietic precursor cells is used to describe the generation of identical and/or more differentiated cells than the precursor cell. The term “hematopoetic growth factor” as used herein refers to a biological molecule that effects the growth and/or development of a hematopoetic cell. Examples of such hematopoetic growth factors include, but are not limited to, G-CSF, GM-CSF, M-CSF, IL-3, TPO and FLT-3.

Other modified chemokines that are useful in the instant invention are variants of these proteins which share the biological activity of the mature (i.e., unmodified) protein. As defined herein, such variants include modified proteins also characterized by alterations made in the known amino sequence of the proteins. Such variants are characterized by having an amino acid sequence differing from that of the mature protein by eight or fewer amino acid residues, and preferably by about five or fewer residues. It may be preferred that any differences in the amino acid sequences of the proteins involve only conservative amino acid substitutions. Conservative amino acid substitutions occur when an amino acid has substantially the same charge as the amino acid for which it is substituted and the substitution has no significant effect on the local conformation of the protein or its biological activity. Alternatively, changes such as the introduction of a certain amino acid in the sequence which may alter the stability of the protein, or permit it to be expressed in a desired host cell, may be preferred. Moreover, variation in primary amino acid sequence with no substantial change in protein structure and function are known in this art. Such variants are readily detected and predicted by algorithms used by those skilled in this art. For example, the well known BLAST algorithm (Altschul, S. F. et al. (1990) J Mol. Biol. 215:403-410; see also http://www.ncbi.nlm.nih.gov/BLAST/) utilizes an amino acid substitution matrix to predict and evaluate tolerable amino acid substitution at residues of the query sequence. Accordingly, the skilled artisan appreciates the scope and meaning of the term “variant” when used to describe equivalent embodiments of a given polypeptide sequence.

The instant polypeptide may also occur as a multimeric form of the mature and/or modified protein useful in this invention, e.g., a dimer, trimer, tetramer or other aggregated form. Such multimeric forms can be prepared by physical association, chemical synthesis or recombinant expression and can contain chemokines produced by a combination of synthetic and recombinant techniques as detailed below. Multimers may form naturally upon expression or may be constructed into such multiple forms. Multimeric chemokines may include multimers of the same modified chemokine. Another multimer may be formed by the aggregation of different modified proteins. Still another multimer is formed by the aggregation of a modified chemokine of this invention and a known, mature chemokine. Preferably, a dimer or multimer useful in the invention would contain at least one desamino chemokine protein and at least one other chemokine or other protein characterized by having the same type of biological activity. This other protein may be an additional desamino chemokine, or another known protein.

A preferred modified chemokine that is useful in the instant invention is a desamino GroB protein. This protein comprises the amino acid sequence of mature GroB protein (SEQ ID NO: 1) truncated at its amino terminus wherein the sequence of the truncated GroB protein (GroB-t) spans amino acids 5 to 73 of the mature protein (SEQ ID NO:2). Also preferred is a variant of the truncated GroB protein wherein one (or more) cysteine residues is (are) added to the amino and/or preferably the carboxy terminus, for example the polypeptide set forth in SEQ ID NO:3.

The instant invention therefore provides a method of enhancing the biological activity of a selected chemokine. This method involves modifying a natively or recombinantly produced chemokine as described herein such that it is covalently bound to a water-soluble polymer. Alternatively, multimers of chemokine molecules may be conjugated to water-soluble polymers. These conjugates may further enhance the biological activity of the resulting composition.

The chemokines, modified chemokines, and variants thereof that are useful in the instant invention may be prepared by any of several methods described below. These polypeptide moieties may be prepared by the solid phase peptide synthetic technique of Merrifield ((1964) J Am. Chem. Soc. 85:2149). Alternatively, solution methods of peptide synthesis known to the art may be successfully employed. The methods of peptide synthesis generally set forth in J. M. Stewart and J. D. Young, “Solid Phase Peptide Synthesis”, Pierce Chemical Company, Rockford, Ill. (1984) or M. Bodansky, Y. A. Klauser and M. A. Ondetti, “Peptide Synthesis”, John Wiley & Sons, Inc., New York, N.Y. (1976) may be used to produce the peptides of this invention.

Modified chemokines may be derived from mature chemokines by enzymatic digestion of the mature chemokine with a suitable enzyme (see, for example, Oravecz, T. et al. (1997) J Exp. Med. 186:1865; Proost, P. et al. (1998) FEBS Letters 432:73; Shioda, T. et al. (1998) PNAS USA 95:6331; and Walter, R. et al. (1980) Mol. Cell. Biochem. 30:111). Moreover, modified amino acids may be incorporated into the growing polypeptide chain during peptide synthesis (M. Hershfield, M. et al. (1991) PNAS88:7185-7189; Felix, A. M. (1997) In J. M. Harris and S. Zalipsky (Eds) Poly(ethylene glycol): Chemistry and Biological Applications. American Chemical Society, Washington, D.C., p218-238). These modified amino acid residues may be chose so as to facilitate covalent conjugation of water-soluble polymers. Also, variant polypeptides may be synthesized wherein amino acid addition, substitution, or deletion are chosen to facilitate subsequent polymer conjugation. Such variant polypeptides may be prepared by chemical synthesis or by recombinant expression. For example, incorporation of additional cysteine residues (by either substitution for existing non-cysteine residues or adding to one or both termini) may be desirable in order to facilitate polymer coupling through the sulfhydryl groups (e.g., Kuan, C. T. et al. (1994) J. Biol. Chem. 269:7610-7616; Chilkoti, A. et al. (1994) Bioconjugate Chem. 5:504-507).

Chemokines that are useful in this invention may preferably be produced by other techniques known to those of skill in the art, for example, genetic engineering techniques. See, e.g., Sambrook et al, in Molecular Cloning, a Laboratory Manual, 2nd edition, Cold Spring Harbor Laboratory, Cold Spring Harbor, N.Y. (1989). Systems for cloning and expression of a selected protein in a desired microorganism or cell, including, e.g. E. coli, Bacillus, Streptomyces, mammalian, insect, and yeast cells, are known and available from private and public laboratories and depositories and from commercial vendors.

Currently, the most preferred method of producing the chemokines of the invention is through direct recombinant expression of the chemokine. For example, the preferred GroB-t protein can be recombinantly expressed by inserting its DNA coding sequence into a conventional plasmid expression vector under the control of regulatory sequences capable of directing the replication and expression of the protein in a selected host cell. See U.S. application Ser. No. 08/557,142, incorporated in its entirety herein by reference.

For recombinant production, host cells can be genetically engineered to incorporate expression systems or portions thereof for chemokines useful in the instant invention. Introduction of polynucleotides encoding chemokines into host cells can be effected by methods described in many standard laboratory manuals, such as Davis et al., Basic Methods in Molecular Biology (1986) and Sambrook et al, in Molecular Cloning, a Laboratory Manual, 2nd edition, Cold Spring Harbor Laboratory, Cold Spring Harbor, N.Y. (1989). Preferred such methods include, for instance, calcium phosphate transfection, DEAE-dextran mediated transfection, transvection, microinjection, cationic lipid-mediated transfection, electroporation, transduction, scrape loading, ballistic introduction or infection.

Representative examples of appropriate hosts include bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

A great variety of expression systems can be used, for instance, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector which is able to maintain, propagate or express a polynucleotide to produce a polypeptide in a host may be used. The appropriate nucleotide sequence may be inserted into an expression system by any of a variety of well-known and routine techniques, such as, for example, those set forth in Sambrook et al. (supra). Appropriate secretion signals may be incorporated into the desired polypeptide to allow secretion of the translated protein into the lumen of the endoplasmic reticulum, the periplasmic space or the extracellular environment. These signals may be endogenous to the polypeptide or they may be heterologous signals.

If the polypeptide is secreted into the medium, the medium can be recovered in order to recover and purify the polypeptide. If produced intracellularly, the cells must first be lysed before the polypeptide is recovered.

Chemokines useful in the instant invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding proteins may be employed to regenerate active conformation when the polypeptide is denatured during isolation and or purification.

Water-soluble polymers that are useful in the instant invention are substantially non-antigenic in order to avoid unwanted immune reactivity towards the composition of the instant invention. Preferred are polyethylene glycol homopolymers, polypropylene glycol homopolymers, polyoxyethylated polyols and polyvinyl alcohol. Suitable polymers may be of any molecular weight. Preferably, the polymers have an average molecular weight between about 1000 and 100,000. More preferred are polymers that have an average molecular weight between about 4000 and 40,000. Polymers suitable for use in the instant invention may be branched, unbranched or star-shaped. Polymers that may be suitable for use in the instant invention are disclosed in the following patents, patent applications and publications: U.S. Pat. Nos. 4,097,470 4,847,325, 5,037,883, 5,252,714, 5,580,853, 5,643,575, 5,672,662, 5,739,208, 5,747,446, 5,824,784, 5,846,951, 5,880,255, 5,919,455, 5,919,758, 5,932,462, 5,985,263, 5,951,974, 5,990,237 6,042,822, 6,046,305, 6,107,272 and 6,113,906; World Patent Publication No. WO 92/16555; European Patent Publication Nos. EP 727,437, EP 727,438, EP 439,508 and EP 714,402; Zalipsky, S. (1995) Bioconjugate Chem 6:150-165; Gregoriadis, G. et al. (1999) Pharma Sciences 9:61-66, each of which is incorporated herein by reference. Moreover, derivatized or functionalized polymers that have been modified in order to facilitate conjugation to polypeptides and other biological substances are suitable for use in the instant invention. For example, modifications of the polymers in order to facilitate conjugation through free amino groups (such as epsilon amino group at lysine residues or a free amino group at the N-terminus), free sulhydryl groups on cysteine residues, or carbohydrate moieties, are desirable. Useful polymers may also include monomethoxy derivatives of polyethylene glycol (mPEG). Most preferred functionalized polymers for use in the instant invention are selected from the group consisting of: methoxy polyethylene glycol succinimidyl propionate; methoxy polyethylene glycol succinimidyl butanoate; succinimidyl ester of carboxymethylated methoxy polyethylene glycol; methoxy polyethylene glycol aldehyde; methoxy polyethylene glycol hydrazide, methoxy polyethylene glycol iodoacetamide; methoxy polyethylene glycol maleimide; methoxy polyethylene glycol tresylate; and methoxy polyethylene glycol orthopyridyl disulfide. The most preferred molecular weight of the aforementioned most preferred functionalized polymers is a member selected from the group consisting of 20,000 daltons and 30,000 daltons.

Conjugation of the chemokines, modified chemokines, and variants thereof that are useful in the instant invention to the water-soluble polymers described herein can be carried out by any of several means that are well known to those skilled in this art.

The chemokine proteins described above can be conjugated to the polymer via either (1) free amine group(s), preferably one or two to minimize loss of biological activity, (2) free carboxyl group(s), preferably one of two to minimize loss of biological activity, (3) free histidine group(s), (4) free sulthydryl group(s) or (5) free thioether group(s) that are either naturally present or genetically engineered into the chemokine molecule and remain free after refolding. The number of polymer molecules that have been conjugated to the protein can be determined by various methods, including, for example, SDS-PAGE gel or size-exclusion chromatography with appropriate molecular markers, matrix-assisted laser desorption and ionization mass spectrometry (MALDI-MS) (Bullock, J. et al. (1996) Anal. Chem. 68:3258-3264), capillary electrophoresis (Kemp, G. (1998) Biotechnol. Appl. Buichem. 27:9-17; Robert, M. J. and Harris, J. M. (1998) J Pharm. Sci. 87:1440-1445). The site of polymer attachment can be determined via digesting the protein into small fragments by an enzyme (e.g., trypsin, Glu-C) and separated by reverse-phase liquid chromatography. A peptide map of the protein before and after the polymer modification would be compared, and fragment with altered elution times sequenced to determine the location(s) of polymer attachments. Alternatively, the polymer can be either fluorescently or radioactively labeled prior to coupling to determine how many moles of the labeled polymer are attached per mole of the protein.

The residue(s) to be conjugated may be: (1) any free amine groups (e.g., epsilon amine group at lysine residue or a free amine group at the N-terminal); (2) free carboxyl groups (e.g., the epsilon carboxylic acid at aspartate or glutamate residues); (3) free imidazole group on histidine; (4) free sulfflydryl groups on cysteine residues, and (5) free thioether groups on methionine that are normally present or genetically engineered into the protein.

The reaction conditions for effecting conjugation further include conducting the above attachment reactions at pH about 6-9, more preferably at pH 6.5-7.5 if the reactive group of the protein is a free amine group, and also to reduce the deamidation reaction which is known to occur at alkaline pH (greater than 7) at asparagine and glutamine residues. Using the above approach, the protein is conjugated via at least one terminal amine-reactive group added to the polymer. These amine-reactive groups include but not limit to: isothiocyanates, isocyanates, acyl azides, N-hydroxysuccinimide (NHS) esters, benzotriazole, imidazole, sulfonyl chlorides, aldehydes, glyoxals, epoxides, carbonates, aryl halides, imidoesters, iodoacetamides, tresylates and anhydrides. The amount of intact activated polymer employed is generally 1- to 10-fold excess over the protein which is in either monomeric or multimeric (preferable dimeric) forms. Generally the reaction process involves reacting the activated polymer with the protein in a 2 to 1 (polymer to protein) ratio. Typically the reaction is carried out in a phosphate buffer pH 7.0, 100 mM NaCl, at 4° C. for from about 1 hr to about 4 hr. Following the conjugation, the desired conjugated protein is recovered and purified by liquid chromatography or the like.

The reaction conditions for effecting conjugation further include conducting the above attachment reactions at pH about 3-9, more preferably are at pH 4-5 if the reactive group of the protein is a free carboxylate group. The carboxyl group on the protein is activated by activation agents such as carbodiimides (e.g., DCC, EDC) or carbonyldiimidazole (e.g., CDI). Using the above approach, the protein is conjugated via at least one nucleophilic functional group added to the polymer. These nucleophilic functional groups include but not limit to: amine or hydrazide. For the above protein, the preferable reaction conditions are at 4° C. and in slightly acidic pH to reduce the deamidation side reaction whch is known to occur at alkaline pH (less than 7) at asparagine and glutamine residues. The amount of intact activated polymer employed is generally 1- to 10-fold excess of the activated polymer over the carobxylated activated protein. Generally the reaction process involves reacting the activated polymer with the protein in a 2 to 1 (polymer to protein) ratio. Typically the reaction is carried out in a MES buffer pH 4.5, at 4° C. for from about 1 hr to about 8 hr. Following the conjugation, the desired conjugated protein is recovered and purified by liquid chromatograhpy or the like.

The reaction conditions for effecting conjugation further include conducting the above attachment reactions at pH about 3-6, more preferably at pH 4-5 if the reactive group of the protein is a free histidine group. Using the above approach, the protein is conjugated via at least one terminal imidazole-reactive group added to the polymer. These imidazol-reactive groups include but not limit to: N-hydroxysuccinimide (NHS) esters and anhydride. The amount of intact activated polymer employed is generally 1-to 1 0-fold excess of the activated polymer over the protein which is in either monomeric or multimeric. Generally the reaction process involves reacting the activated polymer with the protein in a 2 to 1 (polymer to protein) ratio. Typically the reaction is carried out in an acetate buffer, pH 4-5, 100 mM NaCl, at 4° C. for from about 2 hr to about 6 hr. Following the conjugation, the desired conjugated protein is recovered and purified by liquid chromatography or the like.

The reaction conditions for effecting conjugation further include conducting the above attachment reactions at pH about 6-9, more preferably at pH 6-7 if the reactive group of the protein is a free thiol group on the cysteine or the thio ether group on the methionine. Using the above approach, the protein is conjugated via at least one terminal thiol-reactive group added to the polymer. These thiol-reactive groups include but not limit to: haloacetyl, maleimide, pyridyl disulfide derivatives, aziridines, acryloyl derivatives, arylating agents. The amount of intact activated polymer employed is generally 1- to 10-fold excess of the activated polymer over the protein which is in either monomeric or multimeric (preferable dimeric) forms. Generally the reaction process involves reacting the activated polymer with the protein in a 2 to 1 (polymer to protein) ratio. Typically the reaction is carried out in a phosphate buffer pH 6.2, 100 mM NaCl, at 4° C. for from about 1 hr to about 10 hr. Following the conjugation, the desired conjugated protein is recovered and purified by liquid chromatograhpy or the like.

Successful conjugation of water-soluble polymers to therapeutic polypeptides has been previously described in U.S. Pat. No. 4,487,325, U.S. Pat. No. 5,824,784 and U.S. Pat. No. 5,951,974, each of which is incorporated herein in its entirety by reference.

In a further aspect, the present invention provides for pharmaceutical compositions comprising a therapeutically effective amount of the composition of the instant invention, in combination with a pharmaceutically acceptable carrier or excipient. Such carriers include, but are not limited to, saline, buffered saline, dextrose, water, glycerol, ethanol, and combinations thereof. The invention further relates to pharmaceutical packs and kits comprising one or more containers filled with one or more of the ingredients of the aforementioned compositions of the invention. Composition of the instant invention may be employed alone or in conjunction with other compounds, such as therapeutic compounds.

The pharmaceutical composition will be adapted to the route of administration, for instance by a systemic or an oral route. Preferred forms of systemic administration include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if a composition of the instant invention can be formulated in an enteric or an encapsulated formulation, oral administration may also be possible. Administration of these compositions may also be topical and/or localized, in the form of salves, pastes, gels, and the like. Other routes of administration could include pulmonary or nasal delivery either using solution or dry power formulation.

The dosage range required depends on the precise composition of the instant invention, the route of administration, the nature of the formulation, the nature of the subject's condition, and the judgment of the attending practitioner. Suitable dosages, however, are in the range of 0.1-1000 ug/kg of subject. Wide variations in the needed dosage, however, are to be expected in view of the variety of compositions available and the differing efficiencies of various routes of administration. For example, oral administration would be expected to require higher dosages than administration by intravenous injection. Variations in these dosage levels can be adjusted using standard empirical routines for optimization, as is well understood in the art.

The present invention may be embodied in other specific forms, without departing from the spirit or essential attributes thereof, and, accordingly, reference should be made to the appended claims, rather than to the foregoing specification or following examples, as indicating the scope of the invention.

All publications including, but not limited to, patents and patent applications, cited in this specification or to which this patent application claims priority, are herein incorporated by reference as if each individual publication were specifically and individually indicated to be incorporated by reference herein as though fully set forth.

### EXAMPLES

The present invention will now be described with reference to the following specific, non-limiting examples.

### Example 1

### Preparation of Truncated GroB

A truncated form of human GroB protein (GroB-t; SEQ ID NO:2), spanning amino acids 5 to 73 of the mature protein (SEQ ID NO:1), was prepared essentially as described in U.S. Pat. Nos. 6,042,821 and 6,080,398, each incorporated herein by reference.

A. Expression of Recombinant GroB-t.

The coding sequence of GroB-t was amplified by polymerase chain reaction (PCR) from a plasmid containing a complimentary DNA sequence using both a forward primer encoding an NdeI site and a reverse primer containing an XbaI site. These resulting PCR product was subdloned into the E. coli LPL-dependent expression vector pEAKn (pSKF301 derivative) between Ndel and XbaI sites. The polypeptide was produced after chemical induction of the LPL promoter in a lysogenic strain of E. coli containing the wild type (ind+) repressor gene (cl+).

B. Solubilization and Refolding of GroB-t Monomer and Dimer

E. coli LW cells, 400 g, were lysed in 4 liters of lysis buffer containing 25 mM sodium citrate pH 6.0, 40 mM NaCl, 2 mM EDTA by two passages through a Microfluidics (model M110Y) homogenizer at 11,000 psi. The cell lysate was centrifuged at 17,000 g (one hour at 4° C.) and the supernatant was discarded. The insoluble truncated GroB (SEQ ID NO:2) in lysate pellet was solubilized in 1.3 liters of buffer containing 50 mm Tris HCl pH 8.0, 2 M guanidine HCl, 20 mM DTT by stirring 2 hours at room temperature. Soluble reduced GroB-t was recovered by centrifugation at 25,000 g and pellet was discarded. Guanidine HCl and DTT were removed from protein solution by exhaustic dialysis against 50 mM sodium citrate pH 6.0 containing 2 mM EDTA. Majority of E. coli proteins were precipitated during dialysis, while reduced GroB-t stayed in solution. Upon centrifugation, GroB-t was greater than 90% pure. GroB-t solution was concentrated to 3 mg/ml (Amicon YM3 membrane) and raised to pH 8.5 with 0.5 M Trizma base. Air oxidation of GroB-t was performed by stirring for 24 hours at 4° C. Formation of monomer and dimer was monitored by Vydac C 18 (Nest) using 20-40% linear gradient of acetonitrile in 0.1% TFA for 30 min.

C. Purification

When monomer and dimer formation reached maximum and no reduced form left, the reoxidation solution was adjusted to pH 6.5 with 10% acetic acid. GroB-t monomer and dimer were captured on Toyopearl SP-650 M equilibrated in 50 mM Mes-Na pH 6.5 (N-Morpholino ethanesulfonate) (Buffer A). The column was washed with 4 liters of buffer A, and eluted with 4 liters of linear gradient of 0 - 0.5 M NaCl in buffer A. GroB-t monomer was eluted during gradient and GroB-t dimer was eluted with 1 M NaCl solution. Fractions containing GroB-t monomer and dimer were pooled separately. Each pool was adjusted to pH 3.0 with 10% TFA solution, applied to Vydac C18 (2.1×25 cm) equilibrated with 0.1% TFA in 10% acetonitrile, and eluted with linear gradient of 10-40% acetonitrile in 0.1% TFA for 30 min. GroB-t monomer was eluted at approximately 27% acetonitrile. GroB-t dimer was eluted at approximately 31% acetonitrile. Fractions containing GroB-t was pooled, lyophilized to dryness to remove acetonitrile and TFA and solubilized in saline solution. Endotoxin level was 0.1 EU/mg.

Typical yield of GroB-t monomer was approximately 2 mg/g of cells and GroB-t dimer was approximately 0.2 mg/g of cells.

D. Characterization

The molecular weight of the GroB-t dimer as determined on nonreducing SDS-PAGE was approximately twice that of truncated GroB monomer.

GroB-t dimer was boiled in 2% SDS with and without 100 mM DTT at pH 6.8 for 5 minutes. In SDS-PAGE, GroB-t dimer migrated as a dimer without DTT and as a monomer after treated with DTT. Upon reduction, both forms migrated to the same spot indicating that GroB-t dimer is a disulfide linked dimer. GroB-t dimer was mixed with saturated solution of sinapinic acid (3,5-dimethoxy-4 hydroxy-cinnamic acid) in 40% acetonitrile and 1% TFA and was anlayzed in matrix-assisted laser desorption/ionization mass spectrometry, which gave the molecular mass of dimer. The molecular weight of GroB-t dimer, as determined by MALD-MS analysis was 15,069 Da (predicted 15,073 Da), while that of GroB-t monomer was 7,536 Da (predicted 7,537 Da). N-terminal sequencing of GroB-t dimer showed that 2-3% of the final products retained the initiatory Met. Disulfide pairing pattern of GroB-t dimer was the same as that of GroB-t (C5-C3 1, C7-C47), however, all pairings were intermolecular rather than intramolecular. Gel filtration analysis and ultracentrifugation sedimentation equilibrium studies in PBS (pH 7.0) showed that GroB-t dimer exhibited reversible assembly of octamer to hexadecamer at 0.25 mg/ml, while GroB-t was a nonconvalent dimer even at 20 mg/ml. Concentration of GroB-t monomer or dimer has been determined by quantitative amino acid analysis.

### Example 2

### Preparation of PEGylated GroB-t

Solid methoxy polyethylene glycol succinimidyl propionate (Shearwater Polymers Inc.) with an average molecular weight of 5000 or 20,000 Daltons was added to a 2.5 mg/mL solution of the GroB-t in Dulbeccu's Phosphate Buffered Saline (DPBS) pH 7.0. NHS MPEG was added to the protein solution at molar ratio of NHS MPEG to protein of 2:1, 4: 1, or 10:1. The reaction was allowed to proceed at 4° C. for 3 hours. At the end of the reaction, excess amount (e.g., 20×) of glycine (0.5 M) was added to quench the reaction, and pH of the reaction mixture was adjusted to pH 4.5 with 3N HCl. At this stage, the reaction mixture consisted mainly of mono-PEGylated-truncated GroB, some di-, tri, and tetra-PEGylated truncated GroB, non-PEGylated truncated GroB, glycine, and reaction by-product: N-hydroxy succinimide.

Physicochemical Characterization

Five analyses were performed to characterize each sample: (1) SDS-PAGE, (2) reverse-phase liquid chromatography, (3) molecular weight determination, (4) N-terminal sequencing and (5) peptide mapping.

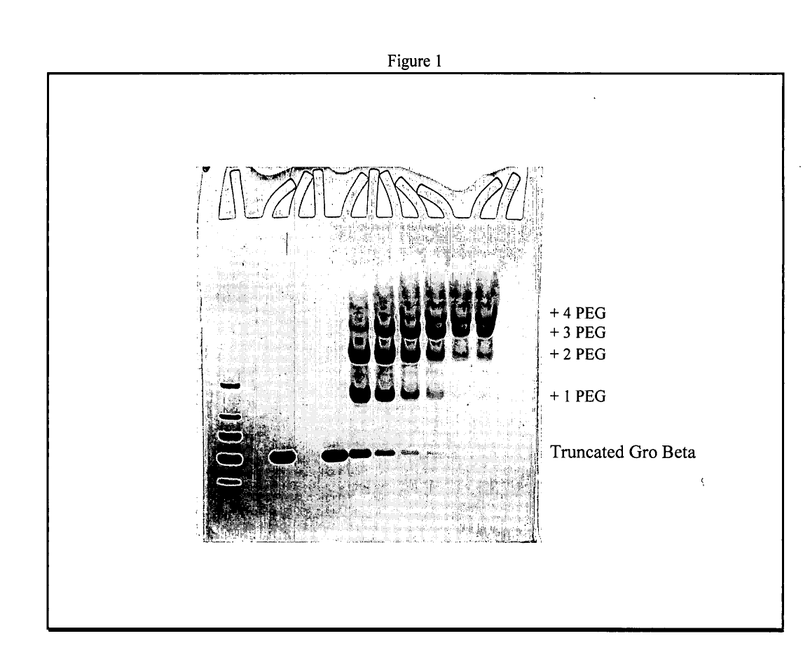

The extent of PEGylation (i.e., the number of PEG molecules attached to a single protein) was analyzed by sodium dodecyl sulfate polyacrylamide gel electrophoresis. Samples of truncated GroB PEGylation reaction mixture were run under reduced conditions at a load of 10.0 ug per lane on 4-12% Bis-Tris polyacrylamide gradient precast gels. Proteins were detected and quantitated after staining with Coomassie R-250. Quantitation was done by laser densitometry. FIG. 1 presents the results of SDS-PAGE analysis of samples obtained from a representative PEGylation experiment. As can be seen, PEGylation reaction conditions can be optimized to control the number of PEG molecules attached per protein molecule.

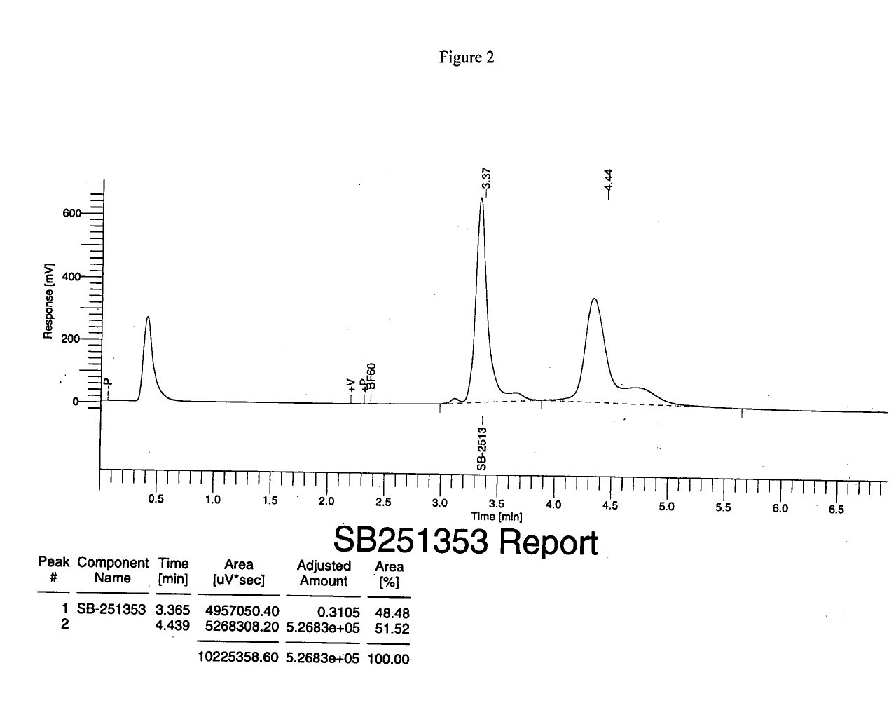

Reverse phase HPLC was used for the quantitation as well as to determine the percent purity of the fractionated PEGylated GroB-t. The assay was performed using a POROS R2/H column with an acetonitrile gradient elution in water and Trifluoroacetic Acid (TFA). UV detection was at 214 nm and the flow rate was 1.5 mL per minute. The column oven temperature is 40° C. and the total assay time was 5.5 minutes. The protein concentration in a sample was calculated based on the total peak area relative to the response of a GroB-t reference standard of known concentration. The protein concentration was reported in mg/mL. A representative HPLC tracing is shown in FIG. 2.

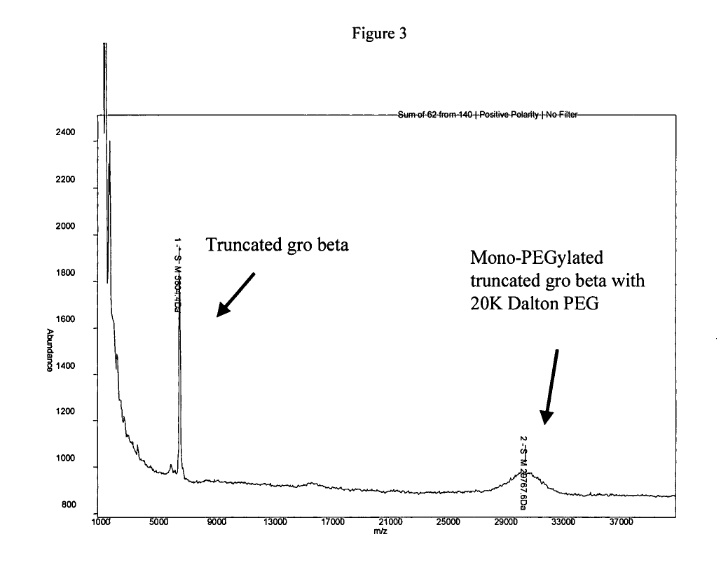

The molecular weight of the various PEGylated protein species was confirmed using MALDI-TOF mass spectrometry. The sample was mixed with a matrix solution, usually sinapinic acid, to obtain final protein concentration within 2-20 picomoles per microliter. The volume of matrix solution has to be equal or greater than the volume of protein sample. 0.7 microliter of such prepared sample was loaded onto the probe and analyzed by MALDI-TOF using an HP G2025A MALDI-TOF mass spectrometer. Peptide standard mixture was prepared and analyzed on the different mesa of the same probe. The instrument was calibrated nased on the masses of peptide standards. Mass of the sample was determined based on this calibration. FIG. 3 provides the results of this analysis on monoPEGylated GroB-t wherein the PEG used for conjugation was MW 20,000 KD PEG.

To verify the location of the attachment of the PEG to the exact location on the protein, purified PEGylated GroB-t samples were analyzed by N-terminal sequencing and peptide mapping. The samples of non-PEGylated and PEGylated GroB-t were diluted with water to the same concentration. Volumes corresponding to 500 picomoles of protein were loaded to the sequencing columns and the samples were sequenced in Hewlett-Packard protein sequencer model G1000A. Initial yield was estimated for each sample based on 10 cycles of the sequence and the yields found for all the samples were compared. The same initial yield was expected based on the same protein load. Any decrease in the initial yield in PEGylated samples was assumed as a result of PEGylation. The results showed that the PEGylated samples had the normal N-terminal sequence for GroB-t but had an approximately 10% lower initial yield than the non-PEGylated control, i.e. indicating approximately 10% PEGylation at the N-terminal amino. The remaining 90% of PEGylation is presumably distributed across the ten lysine side-chains. The 10% modification is roughly the expected amount for a statistical (random) distribution across all the eleven amino groups in the protein.

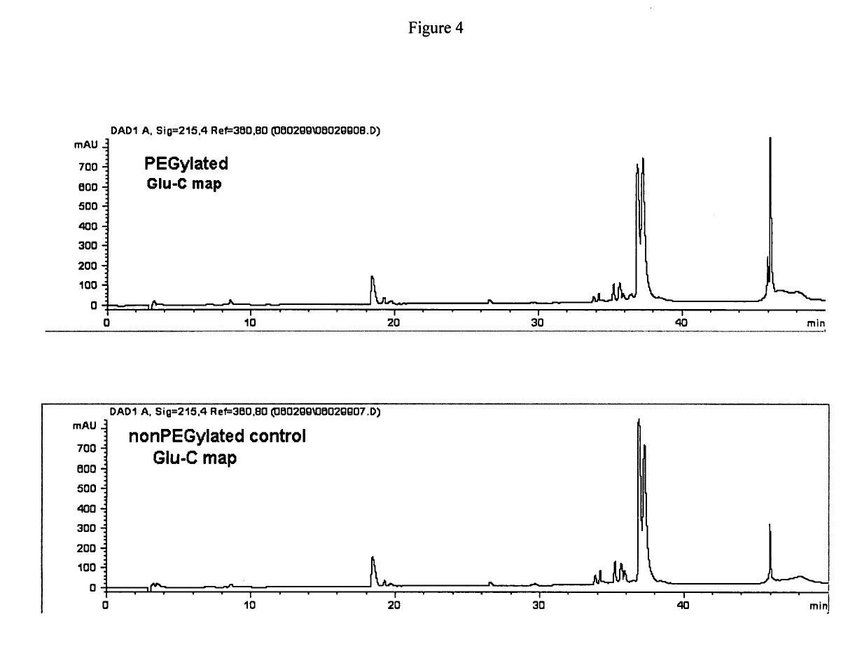

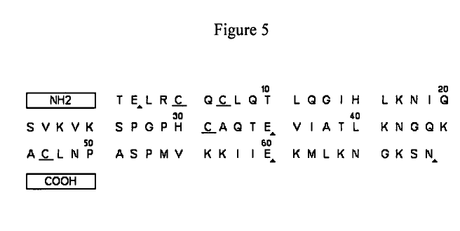

To investigate location of PEGylation at the individual sites or, at least, within separate regions of the sequence, peptide mapping was conducted using Glu-C as well as Trypsin digestion methods (see FIG. 4). As a result of Glu-C digestion, four predicted peptide fragments could be generated: amino acid residues 1-2, 3-35, 36-60 and 61-69 (see FIG. 5).

Note that from FIG. 4, the Glu-C map of the PEGylated protein (upper panel) is only slightly different from that of the nonPEGylated control (lower panel). The relative areas of the main peaks appear to be slightly altered and there is an additional, very hydrophobic peak at about 46 minutes. Since this additional peak(s) was eluting in “cleaning step” portion of the gradient program (80% acetonitrile isocratic), an extrapolation from the standard gradient program to the 80% acetonitrile level was made in order to analyze this additional peak. By doing so, a broad peak eluted at 28 min and found, by N-terminal sequencing, to contain roughly equimolar amounts of three predicted sequences for the Glu-C peptides, 3-35, 36-60, and 61-69. Thus, it seems that these three monoPEGylated Glu-C peptides elute within this same broad peak. It is also possible that the N-terminally modified 1-2 peptide is also present in this fraction but is “blocked” to Edman degradation. The MALDI-TOF MS of this fraction gave broad peaks consistent with PEGylation (data not shown).

The theoretical masses of the eluted peaks, assuming MW_{avg} of 5500 for PEG₅₀₀₀, are presented in Table 1:

TABLE 1

| Glu-C peptide | MW (peptide only) | MW (peptide + PEG) |
|---|---|---|
| 1-2 | 248.1 | 5730 |
| 3-35 | 3656.9 | 9157 |
| 36-60 | 2666.5 | 8167 |
| 61-69 | 1018.6 | 6519 |

The observed masses seem reasonably in agreement with the above theoretical values except for an apparent lack of signal for PEGylated 1-2 and 61-69 peptides. The overall profile is fairly similar to the nonPEGylated control. This is a qualitative indication that the PEGylation must be fairly evenly distributed across the primary amino groups.

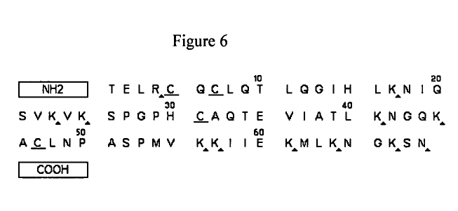

A similar analysis was performed after digestion with trypsin. As a result of trypsin digestion, 11 predicted peptide fragments could be generated: amino acid residues 1-4, 5-23, 18-25, 24-41, 26-45, 42-56, 46-57, 57-61, 58-64, 62-67 and 65-69 (see FIG. 6). The trypsin mapping data differ from the Glu-C mapping in that the PEGylation sites (lysines) are not internal residues in the peptide fragment but, instead, coincide with tryptic cleavage sites (i.e., lysines and arginines). Following a similar analysis as described above, the trypsin mapping data essentially lead to a similar conclusion as that of Glu-C mapping: the PEGylation is evenly distributed at the different amino groups although not in a perfectly random fashion.

### Example 3

### In Vivo Neutrophil Response Assay in Mice

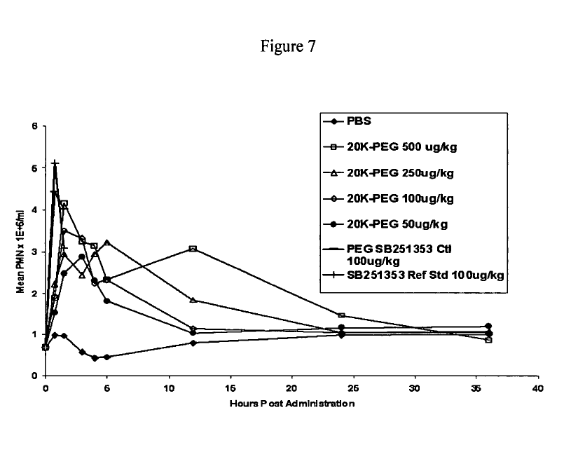

20K PEGylated GroB-t (GroB-t conjugated to one 20K PEG molecule attached randomly to a lysine residue) was evaluated in normal B6D2F-1 mice. A single subcutaneous injection of 20K PEGylated GroB-t prepared as described above was administered to mice at doses of 500, 250, 100, or 50 ug/kg. Unmodified GroB-t (100 ug/kg) or PBS were injected as controls. Groups of mice (4 per time point per dose) were bled by cardiac puncture at various time points post injection. Control GroB-t groups were bled at 45 and 90 minute time points. Results are shown in FIG. 7. Injection of 20K PEGylated GroB-t significantly increased neutrophil counts at all doses administered from 50 ug/kg up to 500 ug/kg. The neutrophil response was delayed in comparison to unmodified GroB-t, however the duration of increased neutrophil counts was significantly prolonged with counts over PBS in all groups 5 hours post dose and 12 hours post dose in the 500 and 250 ug/kg groups (see Table 2 below).

TABLE 2

| | Neutrophil Counts (×10⁻⁶/ml) ± SD at | | | | | | | |
|---|---|---|---|---|---|---|---|---|
| Treatment | 0.75 hr | 1.5 hr | 3 hr | 4 hr | 5 hr | 12 hr | 24 hr | 36 hr |
| PBS | 0.98 ± 0.1 | 0.96 ± 0.06 | 0.58 ± 0.06 | 0.42 ± 0.05 | 0.45 ± 0.07 | 0.79 ± 0.13 | 0.98 ± 0.13 | 1.0 ± 0.14 |
| 20K-PEG | 2.23 ± 0.8 | 2.94 ± 0.37 | 2.44 ± 0.23 | 2.94 ± 0.13 | 3.23 ± 0.36 | 1.84 ± 0.21 | 1.04 ± 0.14 | 1.08 ± 0.16 |
| 250 ug/kg | | | | | | | | |
| 20K-PEG | 1.89 ± 0.06 | 4.17 ± 0.29 | 3.24 ± 0.51 | 3.15 ± 0.84 | 2.32 ± 0.15 | 3.07 ± 0.34 | 1.46 ± 0.17 | 0.86 ± 0.13 |
| 500 ug/kg | | | | | | | | |

### Example 4

### Increased Neutrophil Bactericidal Activity upon Administration of 20K PEGylated GroB-t

20K PEGylated GroB-t was evaluated in normal B6D2F-1 mice. A single subcutaneous injection of 20K PEGylated GroB-t prepared as described above was administered to mice at a dose of 500 ug/kg. Unmodified GroB-t (100 ug/kg) or PBS were injected as controls. Groups of mice (4 per time point per dose) were bled by cardiac puncture at various time points post injection. Neutrophils were enumerated via a H-1 Technicon hematology analyzer equipped with veterinary software.

Bactericidal activity was determined by incubating fresh blood (200 ul) with 20 ul of a solution of Staphlococcus aureus (6-8×10⁸ CFU/ml) for 2 hours at 37° C. One hundred microliters of this mixture were treated to lyse blood cells and the resulting solution transferred to bacteriologic agar plates. Staphlococcus aureus colonies were enumerated after 24 hours of incubation. Percent killing was calculated based on the reduction of CFU compared to media-treated (i.e., Staphlococcus aureus incubated with media) controls.

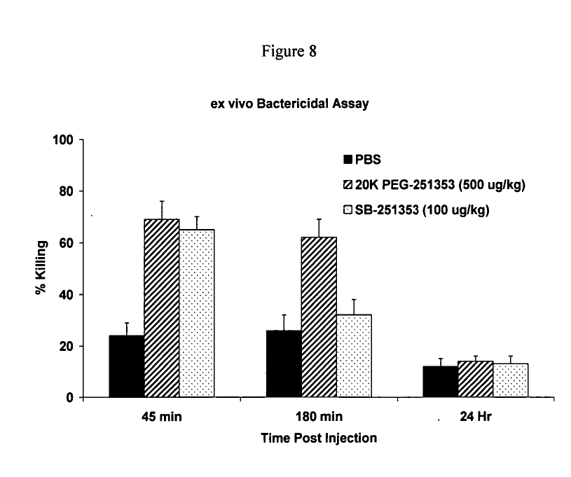

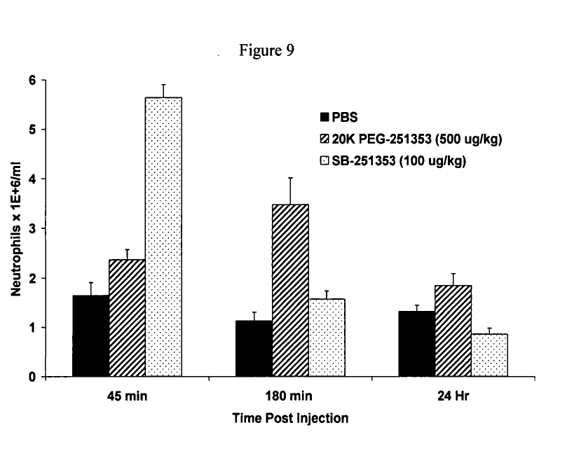

Administration of 20K PEGylated GroB-t resulted in increased neutrophil bactericidal activity at 45 minutes post injection, and activity remained elevated at 180 minutes post single subcutaneous injection. In contrast, although administration of unPEGlyated GroB-t resulted in increased bactericidal activity at 45 minutes post injection, bactericidal activity returned to normal at 180 minutes post administration (see FIG. 8). Surprisingly, the increased bactericidal activity of 20K PEGylated GroB-t observed at 45 minutes was comparable to unPEGlyated GroB-t despite no substantial increase in neutrophils (see FIG. 9); i.e., neutrophils from 20K PEGylated GroB-t-treated animals are more efficient killers of Staphlococcus aureus than neutrophils obtained from animals treated with unPEGlyated GroB-t. These data indicate that 20K PEGylated GroB-t has an unexpected increase in bactericidal activity without the concomitant elevation numbers of neutrophils.

### Example 5

### Improved Pharmacokinetics, Including Improved Subcutaneous Bioavailability of 20K PEGylated GroB-t in Rats

Three or four male Sprague-Dawley rats (weighing approximately 275-600g) were used for each treatment group. The animals were housed in clear PVC boxes with wire lids in unidirectional air flow rooms with controlled temperature (22±2° C.), humidity (50±10%) and 12 hour light/dark cycles. Rats were acclimatized for at least 5 days prior to the experiment, and provided food (Certified Rodent Chow #5001, Purina Mills Inc., St. Louis, Mo.) and filtered tap water ad libitum.

For intravenous dosing, drug (20K PEGylated GroB-t ) was administered through a tail vein. The dose was delivered in less than 15 sec and in a volume of less than 5 mL/kg. Intravenous dosing was followed with a 0.9% saline flush (0.1 mL). For subcutaneous dosing, drug was administered under the skin at the scruff of the neck. The total dose administered was approximately 0.5 mg/kg for all treatment groups. Blood samples were collected pre-dose and at various times following administration for up to 72 hours post-dose. Blood samples were collected by lateral tail vein stick (avoiding the dosing vein for the first hour) into labeled polypropylene tubes containing anticoagulant. Plasma was collected by centrifugation, frozen on solid carbon dioxide and stored at −20° C. or below prior to analysis.

Sensitive and selective enzyme-linked immunosorbent assays were developed for the determination of GroB-t and PEGylated GroB-t in rat plasma. In these assays, drug was captured on a microtiter plate with a Gro-specific monoclonal antibody and the complex was detected with GroA-specific polyclonal antibody (reagents available from R&D Systems, Minneapolis Minn.). Concentrations were interpolated from freshly prepared calibration curves using the appropriate analyte. Also, quality control samples were prepared by spiking control plasma at various concentrations with GroB-t or PEGylated GroB-t. These were stored and analyzed with authentic samples and used to assess day to day assay performance.

Non-compartmental pharmacokinetic analysis of plasma concentration-time data was performed. The following pharmacokinetic parameters were determined: maximum observed plasma concentration (Cmax), time to Cmax (Tmax), area under the plasma concentration-time curve from time zero to infinity (AUC(0-inf)) and terminal phase half-life (T½). The subcutaneous bioavailability was estimated by dividing the mean AUC(0-inf) obtained after subcutaneous dosing by the mean AUC(0-inf) obtained after intravenous dosing for each drug.

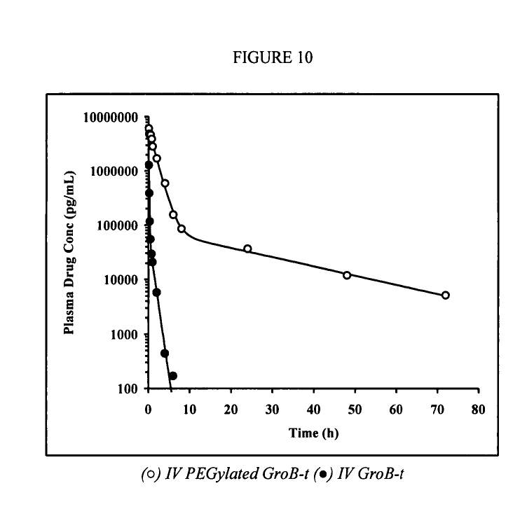

Consistent with its impact on the neutrophil count versus time profile in the mouse, conjugation of GroB-t with PEG dramatically increased the half-life and decreased the clearance of the chemokine relative to the unmodified protein (Table 3; FIG. 10). Specifically, pegylation resulted in a greater than 10-fold increase in terminal half-life (T½) and a greater than 30-fold increase in area under the plasma concentration-time curve (AUC).

TABLE 3

| | Cmax | Tmax | AUC(0-inf) | Term T½ | F |
|---|---|---|---|---|---|
| Group | (ug/mL) | (h) | (ug · h/mL) | (h) | (%) |
| PEGylated | | | | | |
| SC | 0.110 | 8 | 3.92 | 24.9 | 35 |
| IV | 6.88 | NA | 11.2 | 15.8 | 100 |
| Unmodified | | | | | |
| SC | 0.023 | 0.89 | 0.040 | 1.8 | 14 |
| IV | 0.760 | NA | 0.278 | 1.0 | 100 |

Abbreviations of mean pharmacokinetic parameters (n = 3-4/group) are as follows: Cmax, maximum observed plasma concentration; Tmax, time of Cmax (* median given); AUC(0-inf), area under the plasma concentration-time curve from time 0 to infinity; Term T½, terminal half-life; F, subcutaneous bioavailability.

Drug bioavailability and drug clearance are independent pharmacokinetic parameters that have separate influences on drug exposure following subcutaneous administration. For example, drug formulation may increase or decrease bioavailability following extravascular administration while drug clearance, for the same active ingredient, is unaltered by changing the formulation. Thus it does not follow trivially that a modification of a chemokine that decreases intravenous drug clearance would be expected, a priori, to increase subcutaneous bioavailability. In fact, subcutaneous bioavailability may be increased or decreased as a result of this modification.

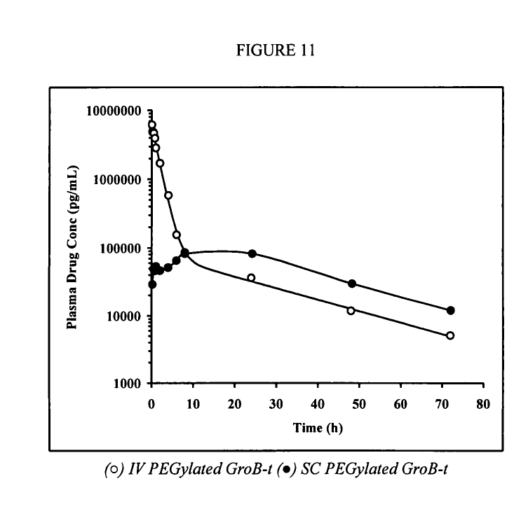

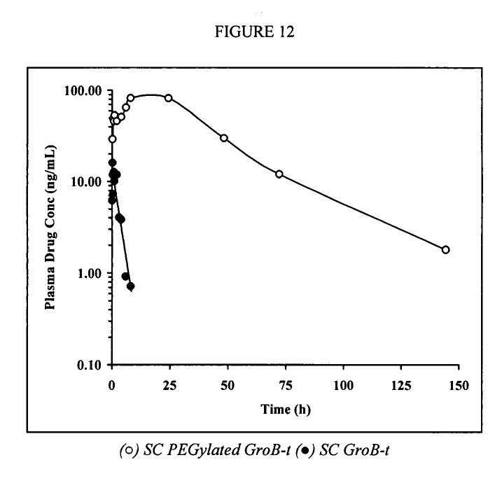

Surprisingly, and in addition to having greatly improved the intravenous pharmacokinetic profile, PEGylation of GroB-t unexpectedly resulted in a greater than 2-fold increase (from 14 to 35%) in the subcutaneous bioavailability (the fraction of the dose absorbed from the subcutaneous injection site into the systemic circulation) of the chemokine (Table 3; FIGS. 11 and 12). This may have been due to increased stability of the drug at the injection site and/or better ability of the conjugate to circumvent barriers to absorption.

The observed improvement in the subcutaneous bioavailability of this chemokine increases the technical feasability of developing a subcutaneous formulation for this product. Relatively less of the pegylated product is lost upon subcutaneous administration within the injection site and more is available to exert systemic effects. Subcutaneous administration is much more convenient and less expensive than intravenous administration. Therefore the improved pharmacokinetic profile following subcutaneous administration is valuable both to the patient and to the manufacturer of the drug.

### Example 6

### Preparation of Truncated GroB with Additional Cysteine Residue at C-terminus

A variant form of human GroB-t protein, GroB-t C-Cys, comprising the GroB-t polypeptide with a cysteine added to the C-terminus (SEQ ID NO:3), was prepared following similar methods as described in U.S. Pat. No. 6,042,821 and U.S. Pat. No. 6,080,398, each incorporated herein by reference.

A. Expression of recombinant GroB-t C-Cys

A DNA fragment encoding GroB-t C-Cys was prepared and inserted into expressed the E. coli expression vector pET22b (Novagen; Cat. No. 70765-3). GroB-t C-Cys was expressed in E. coli strain BL21(DE3), also obtained from Novagen (Cat. No. 70235-3). Recombinant cells were grown at 37° C. to mid-log phase in LB medium supplemented with 50 ug/ml ampicillin and 2% glucose. Expression was induced by addition of 1 mM IPTG, and cells were harvested 2 hours later.

B. Cell Lysis, Refolding, and Purification of GroB-t C-Cys

Frozen cells were dispersed in 50 mM sodium citrate buffer, pH 6.0, containing 40 mM NaCl, 5% glycerol and 2 mM EDTA (10 m/lg of cells) and lysed by two passages through a Microfluidics M110Y or Gaulin at 10,000 psi. The lysate was centrifuged at 17000 g for one hour at 4° C. All of GroB-t C-Cys was contained in the resulting pellet; accordingly, the supernate was discarded. The pellet was washed with lysis buffer (2ml/g cells) and solubilized in 2M Guanidine HCl, 50 mM Tris HCl 2mM EDTA pH 8.0 (2 ml/g of cells) for two hours at 25° C. The solution was diluted in an equal volume of water and insoluble material was removed by centrifugation at 15000 g for one hour. In order to convert all GroB-t C-Cys to the reduced form, the supernate was adjusted to 40 mM DTT and was incubated overnight at 4° C. The solution was diluted to 10 ml/g of cells with 5 mM HCl, which resulted in mass precipitation. The precipitate (contained no GroB-t C-Cys) was removed by centrifugation at 5000 g for 30 min. The clear supernatant was dialyzed (3K cutoff) or diafiltered (Filtron 3K cutoff) against 1 mM HCl. The reduced GroB-t C-Cys in 1 mM HCl was diluted (30ml/g cells), neutralized to pH 7.5 with 2 M Trizma base, and adjusted to 1 mM glutathione, 0.2mM oxidized glutathione, and 1 mM EDTA. Reoxidation was allowed for approximately 18 hours at 25° C. The solution was adjusted to pH 6.5 with 1 M HAc and applied to Toyopearl SP 650 M column (2 ml resing of cells) equilibrated with 25 mM MES buffer at pH 6.5 (Buffer A). The column was washed with 5 column volumes of Buffer A and eluted with a 6 column volume linear gradient to 1 M NaCl in buffer A. The pool was passed through Q-Sepharose in 0.4 M NaCl in order to remove any associated DNA or endotoxin, dialyzed in 1 mM potassium phosphate pH 6.5 (Buffer P) containing 50 mM NaCl, and then applied to a hydroxyapatite (HA) column (BioRad Macro-Prep Ceramic Hydroxyapatite Type I). The HA column was washed with 0.15 M NaCl in Buffer P to remove impurities, and GroB-t C-Cys was eluted with 0.5 M NaCl in Buffer P. The HA pool was dialyzed against saline and stored at −70° C., where it was stable indefinitely.

To obtain homogeneous GroB-t C-Cys, the pool from the Toyopearl SP 650 M column was fractionated using C 18 RP-HPLC column instead of Q-Sepharose and HA columns. The SP pool was adjusted to 0.1% TFA and applied to Vydac C4 (2.2×25 cm, 95 ml, 10 micron, Nest Group) which was equilibrated with 5% Buffer B (80% acetonitrile in 0.1% TFA). The column was washed with 2.5 column volumes of 5% Buffer B. GroB-t C-Cys was eluted with a 6 column volume linear gradient to 50% Buffer B. The pool from the C4 column was lyophilized to dryness, resuspended to 3 mg/ml in 1 mM HCl to avoid dimer formation, and stored at −80° C. before use.

C. Preparation of PEGylated GroB-t C-Cys

The GroB-t C-Cys solution (3 mg/ml in 1 mM HCl, pH 3.0) was added dropwise to a Dulbecco's Phosphate Buffered Saline (DPBS) at pH 7.0, containing pre-dissolved methoxy polyethylene glycol maleimide (MAL MPEG; Shearwater Polymers Inc.) with an average molecular weight of 20,000 to 40,000 Daltons. The molar ratio of MAL MPEG to protein was 2:1 or 4:1. The reaction was allowed to proceed at 4° C. for 24 hours. At the end of the reaction, an excess amount (e.g.,10×) of cysteine (0.5 M) was added to quench the reaction. At this stage, the reaction mixture consisted mainly of mono-PEGylated- GroB-t C-Cys and non-PEGylated GroB-t C-Cys.

What is claimed is:

1. A biologically active composition comprising a polypeptide covalently conjugated to a water-soluble polymer wherein the polypeptide is a chemokine, a modified chemokine, or a biologically active derivative or variant thereof.

2. The composition of claim 1 wherein the chemokine is a CXC chemokine.

3. The composition of claim 2 wherein the CXC chemokine is GroB.

4. The composition of claim 3 wherein the CXC chemokine is a truncated form of GroB.

5. The composition of claim 4 wherein the truncated form of GroB comprises GroB-t as set forth in SEQ ID NO:2.

6. The composition of claim 1 wherein the chemokine is a variant of GroB.

7. The composition of claim 6 comprising the polypeptide set forth in SEQ ID NO:3.

8. The composition of claim 1 wherein the water-soluble polymer is a member selected from the group consisting of polyethylene glycol homopolymers, polypropylene glycol homopolymers, poly(N-vinylpyrrolidone), poly(vinyl alcohol), poly(ethylene glycol-co-propylene glycol), poly(N-2-(hydroxypropyl)methacrylamide), and pol(sialic acid).

9. The composition of claim 8 wherein the water-soluble polymer is unsubstituted.

10. The composition of claim 8 wherein the water-soluble polymer is substituted at one end with an alkyl group.

11. The composition of claim 8 wherein the water-soluble polymer is a polyethylene glycol homopolymer.

12. The composition of claim 11 wherein the polyethylene glycol homopolymer is linear.

13. The composition of claim 11 wherein the polyethylene glycol homopolymer is branched.

14. The composition of claim 11 wherein the polyethylene glycol homopolymer is star-shaped.

15. The composition of claim 12 wherein the polyethylene glycol homopolymer is monomethoxy-polyethylene glycol.

16. The composition of claim 13 wherein the polyethylene glycol homopolymer is monomethoxy-polyethylene glycol.

17. The composition of claim 14 wherein the polyethylene glycol homopolymer is monomethoxy-polyethylene glycol.

18. The composition of claim 1 wherein the composition is PEGylated GroB-t.

19. The composition of claim 1 wherein the composition is PEGylated GroB-t C-Cys.

20. The composition of claim 1 further comprising a second biologically active composition comprising a hematopoetic growth factor.

21. The composition of claim 18 wherein the hematopoetic growth factor comprises a member selected from the group consisting of G-CSF, GM-CSF, M-CSF, IL-3, TPO and FLT-3.

22. The composition of claim 18 wherein the hematopoetic growth factor comprises a member selected from the group consisting of an IL-3 mutein and a TPO mutein.

23. The composition of claim 18 wherein the hematopoetic growth factor is conjugated to a water-soluble polymer.

24. A method of treating myelosuppression in a patient by administering an effective dose of the composition of claim 1.

25. A method of enhancing the microbicidal activity of phagocytic cells in a subject by administering an effective dose the composition of claim 1.

26. A method of mobilizing hematopoietic stem cells of a subject by administering an effective dose of the composition of claim 1.

27. A method of treating chemotherapy- or irradiation-induced cytopenia in a patient by administering an effective dose of the composition of claim 1.

28. A method of preventing chemotherapy- or irradiation-induced cytopenia in a patient by administering an effective dose of the composition of claim 1 to the patient before or during chemotherapy or irradiation.

29. A method of preparing a biologically active composition comprising
a) obtaining a chemokine or a biologically active variant or derivative thereof;
b) contacting the chemokine and or biologically active variant or derivative with functionalized water-soluble polymer.

30. The method of claim 29 wherein the funtionalized water soluble polymer is a member selected from the group consisting of methoxy polyethylene glycol succinimidyl propionate, MW 20,000; methoxy polyethylene glycol succinimidyl propionate, MW 30,000; methoxy polyethylene glycol succinimidyl butanoate, MW 20,000; succinimidyl ester of carboxymethylated methoxy polyethylene glycol, MW 20,000; methoxy polyethylene glycol aldehyde, MW 20,000; methoxy polyethylene glycol aldehyde, MW 30,000; methoxy polyethylene glycol hydrazide, MW 20,000; methoxy polyethylene glycol maleimide, MW 20,000; methoxy polyethylene glycol maleimide, MW 30,000; methoxy polyethylene glycol orthopyridyl disulfide, MW 20,000; methoxy polyethylene glycol orthopyridyl disulfide, MW 30,000; methoxy polyethylene glycol iodoacetamide, MW 20,000; and methoxy polyethylene glycol iodoacetamide, MW 30,000.

31. The method of claim 29 wherein the chemokine is a CXC chemokine.

32. The method of claim 31 wherein the CXC chemokine is GroB.

33. The method of claim 32 wherein the CXC chemokine is a truncated form of GroB.

34. The method of claim 33 wherein the truncated form of GroB is GroB-t as set forth in SEQ ID NO:2.

35. The product of the method of claim 29.

36. A method of improving the pharmacokinetics of a chemokine comprising the step of conjugating the chemokine to a water-soluble polymer.

37. The method of claim 36 wherein the chemokine is a CXC chemokine.

38. The method of claim 37 wherein the CXC chemokine is GroB.

39. The method of claim 38 wherein the CXC chemokine is a truncated form of GroB.

40. The method of claim 36 wherein the water-soluble polymer is a polyethylene glycol homopolymer.

41. The method of claim 36 wherein the intravenous bioavailability is improved.

42. The method of claim 36 wherein the subcutaneous bioavailability is improved.
